(19) United States
(12) Patent Application Publication
(10) Pub. No.: US 2022/0152336 A1
(43) Pub. Date: May 19, 2022

(54) Portable Breathing System, Method and Apparatus for Removing Airborne Pathogens and Particulates

(71) Applicant: Kim Christensen, Livermore, CA (US)

(72) Inventor: Kim Christensen, Livermore, CA (US)

(21) Appl. No.: 17/530,448

(22) Filed: Nov. 18, 2021

Related U.S. Application Data
(60) Provisional application No. 63/115,437, filed on Nov. 18, 2020.

(51) Int. Cl. A61M 16/10; A61M 16/06

(52) U.S. Cl. CPC A61M 16/1055; A61M 16/06

(57) ABSTRACT

A system and method of removing pathogens and particulates in a breathable air supply includes supplying pressurized air from a portable pressurized air source, flowing the pressurized air through a filter media including capturing multiple pathogens and particulate contaminates from the pressurized air, outputting filtered, pressurized air and delivering the filtered, pressurized air to a mask.

### CROSS REFERENCE TO RELATED APPLICATIONS

This application claims priority from U.S. Provisional Patent Application No. 63/115,437 filed on Nov. 18, 2020 and entitled “Portable Breathing System, Method and Apparatus for Removing Airborne Pathogens and Particulates,” which is incorporated herein by reference in its entirety for all purposes.

### FIELD OF THE DISCLOSURE

The present disclosure relates generally to breathing masks, and more particularly, to portable, wearable systems, methods and apparatus for a filtering pathogens and particulates from air before inhalation.

### BACKGROUND

There are many types of particulate breathing masks for filtering particles from air before being inhaled by the person wearing the mask. However, most such particulate breathing masks are significantly compromised in their ability to remove smaller particles, such as airborne pathogens including bacteria and virus particles. The filter material of the masks are selected to compromise the size of particles removed from the air and restricting the air flow to improve the wearer's comfort and ease of breathing. Restated, as the particle size removed becomes smaller, the airflow through the mask becomes more restricted. The restricted airflow often stifles the wearer's ability to breathe.

It is in this context that the following embodiments arise.

### SUMMARY

Broadly speaking, the present disclosure fills these needs by providing a portable, powered, wearable system, method and apparatus for removing pathogens and particulates in an air supply. It should be appreciated that the present disclosure can be implemented in numerous ways, including as a process, an apparatus, a system, computer readable media, or a device. Several inventive embodiments of the present disclosure are described below.

One implementation includes a method of removing pathogens and particulates in an air supply including supplying pressurized, the airflow being provided by a portable, pressurized air source, flowing the pressurized air through a filter media. The filter media capturing multiple pathogens and particulate contaminates from the pressurized air to output filtered, pressurized air. Delivering the filtered, pressurized air to a face mask, the filtered, pressurized air having a pressure equal to or greater than atmospheric pressure within the face mask, the face mask forming a substantially air tight seal around a wearer's mouth and nose.

Another implementation includes a system for removing pathogens and particulates in a portable air supply including a portable pressurized air source, a filter fluidly coupled to the portable pressurized air source. The filter capable of capturing multiple pathogens and particulate contaminates from a pressurized air flow output from the portable pressurized air source. The filter media outputting filtered, pressurized air flow. The system further including a mask fluidly coupled to the filter, the filtered, pressurized air having a pressure equal to or greater than atmospheric pressure within the face mask, the face mask capable of forming a substantially air tight seal around a wearer's mouth and nose.

Other aspects and advantages of the disclosure will become apparent from the following detailed description, taken in conjunction with the accompanying drawings, illustrating by way of example the principles of the disclosure.

### BRIEF DESCRIPTION OF THE DRAWINGS

The present disclosure will be readily understood by the following detailed description in conjunction with the accompanying drawings.

FIG. 1 is a schematic diagram of a filtered air system for removing pathogens and particulates in a portable air supply, for implementing embodiments of the present disclosure.

FIG. 2 is a block diagram of the filtered air system for removing pathogens and particulates in a portable air supply, for implementing embodiments of the present disclosure.

FIG. 3 is a pictorial view of the filtered air system for removing pathogens and particulates in a portable air supply, for implementing embodiments of the present disclosure.

FIG. 4 is a pictorial view another implementation of the filtered air system for removing pathogens and particulates in a portable air supply, for implementing embodiments of the present disclosure.

FIG. 5 is a flowchart diagram that illustrates the method operations performed in removing pathogens and particulates in a portable air supply, for implementing embodiments of the present disclosure.

FIGS. 6 and 7 are more detailed pictorial views of an example pump, for implementing embodiments of the present disclosure.

FIG. 8A is an example mask 120′, for implementing embodiments of the present disclosure.

FIG. 8B is a side view of another example mask 120″, for implementing embodiments of the present disclosure.

FIG. 9 is a graph showing the airflow rate during a wearer's typical inhalation and exhalation, for implementing embodiments of the present disclosure.

FIG. 10 is an example filter, for implementing embodiments of the present disclosure.

FIG. 11 is an optional low battery alarm circuit, for implementing embodiments of the present disclosure.

FIGS. 12 and 13 are an example check/pressure relief valve, for implementing embodiments of the present disclosure.

FIGS. 14A and 14B are an example mask, for implementing embodiments of the present disclosure.

FIG. 15 is an optional, lighted, power switch, for implementing embodiments of the present disclosure.

### DETAILED DESCRIPTION

Several exemplary embodiments for systems, methods and apparatus for removing pathogens and particulates in a portable air supply before inhalation of the air, will now be described. It will be apparent to those skilled in the art that the present disclosure may be practiced without some or all of the specific details set forth herein.

Typical prior art particulate face masks are passive filtering devices primarily relying on a lower pressure formed between the mask and the wearer's face that occurs when the wearer inhales a breath. Typical prior art particulate face masks do not remove small particles such as pathogens e.g., bacteria and virus particles as such a filter media would restrict the airflow to a level that would cause significant difficult in inhaling breath, discomfort and increase anxiety of the wearer.

The following described embodiments include a pressurized air source than forces pressurized air through a filter media capable of removing at least 99.9 percent of the desired pathogens at an airflow rate and/or a pressure that does not restrict the wearer's inhalation of breath.

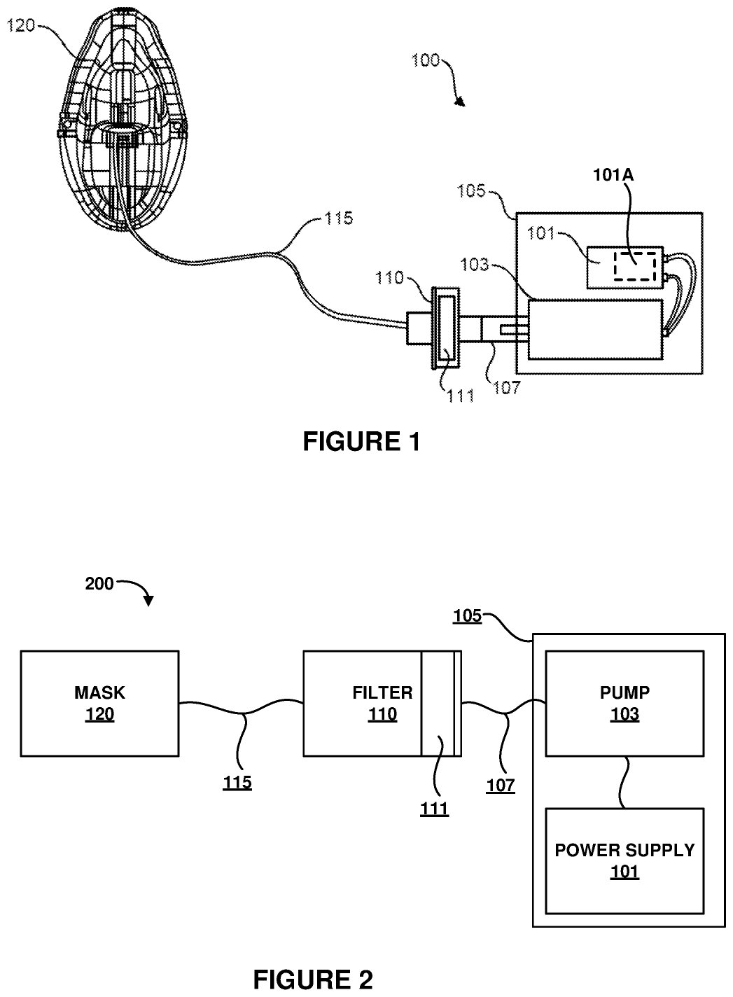

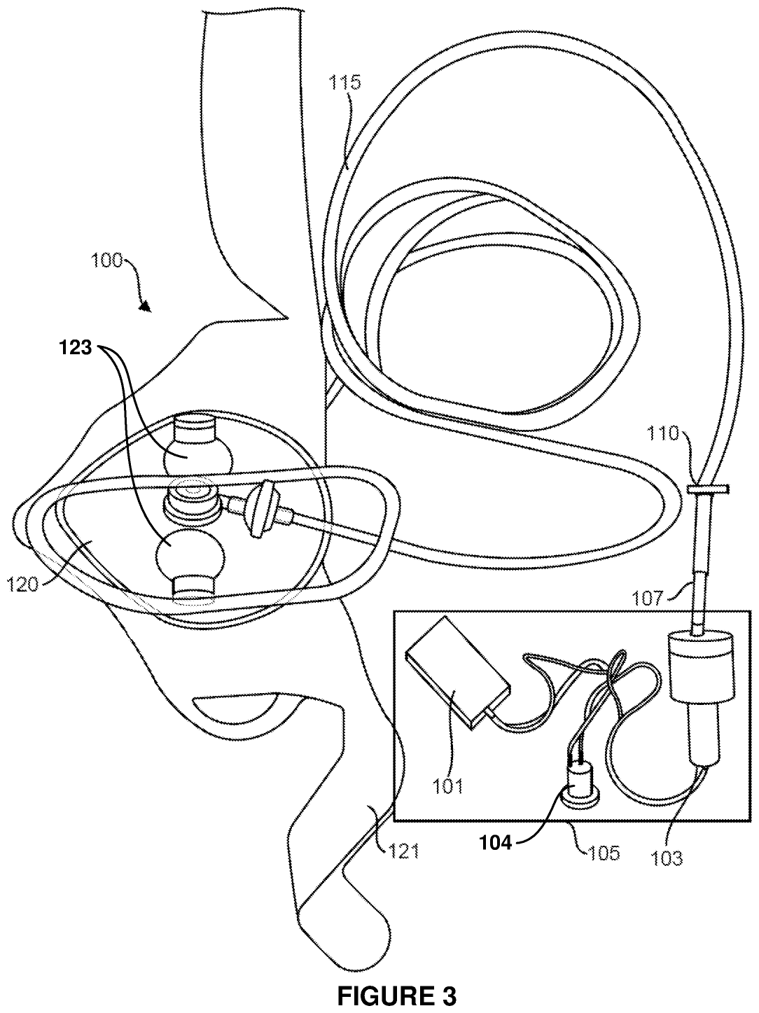

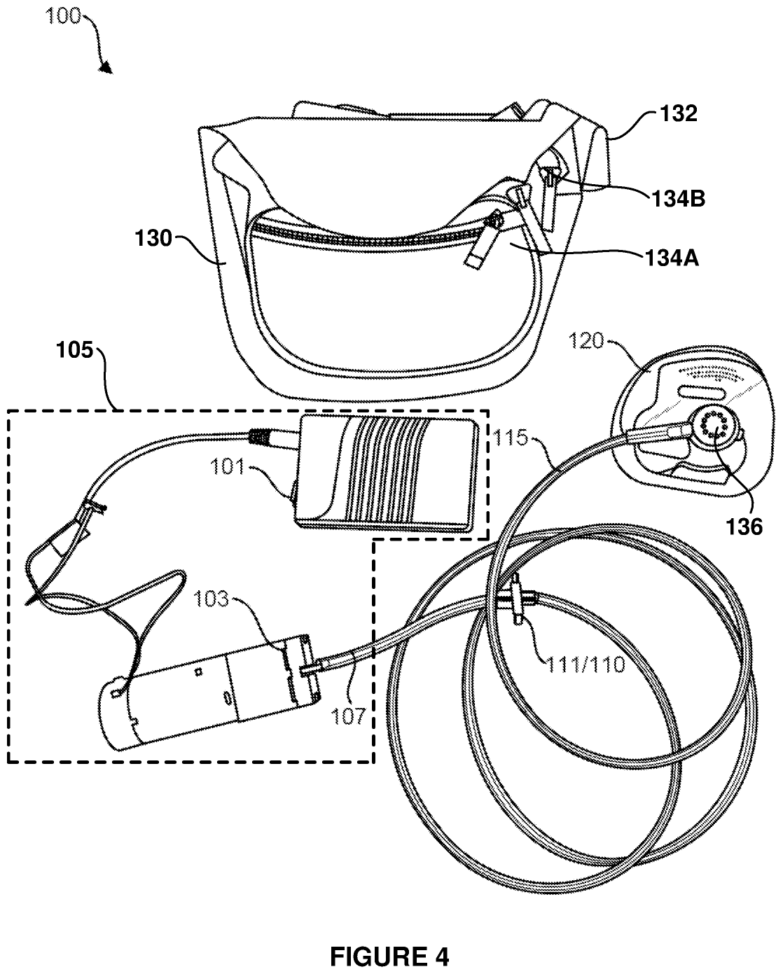

FIG. 1 is a schematic diagram of a filtered air system 100 for removing pathogens and particulates in a portable air supply, for implementing embodiments of the present disclosure. FIG. 2 is a block diagram 200 of the filtered air system 100 for removing pathogens and particulates in a portable air supply, for implementing embodiments of the present disclosure. FIG. 3 is a pictorial view 300 of the filtered air system 100 for removing pathogens and particulates in a portable air supply, for implementing embodiments of the present disclosure. FIG. 4 is a pictorial view another implementation of the filtered air system 100 for removing pathogens and particulates in a portable air supply, for implementing embodiments of the present disclosure.

The filtered air system 100 includes a portable pressurized air source 105, a filter 110 and a mask 120. The portable pressurized air source 105 is fluidly coupled to the filter 110 by a first conduit 107. The filter 110 is fluidly coupled to the mask 120 by a second conduit 115. The first conduit or the second conduit can also include a one-way valve to make sure the air only flows toward and into the mask. The filter 110 can be any suitable filter capable of capturing the desired pathogens from the air flowing through the filter.

The filter 110 includes filter media 111. The filter media is approved by the FDA (Food and Drug Administration) and is certified as anti-viral and/or anti-bacterial in that the filter media is capable of capturing at least 99.9 percent of bacteria and virus particles, respectively. A virus generally needs a carrier, such as a microscopic, or larger, water droplet or a bacterium. The filter media 111 can have any suitable porosity for removing the desired pathogen and particle size. In at least one implementation, the filter media 111 has a porosity of about 1.2 microns. It should be understood that the porosity of the filter media 111 can be more or less than 1.2 micron, as may be desired by the wearer. The porosity of the filter media 111 can correlate to the service life of the filter 110 and the corresponding airflow rates. By way of example, a high airflow rate with a larger porosity will have a longer filter service life but a less effective filtering function than a smaller porosity filter in the same higher airflow rate.

The pressurized air source 105 can include a pressurized air bottle (not shown) filled with pressurized air. The pressurized air source 105 can additionally or alternatively include a pump 103 and a portable power supply 101 to pressurize ambient air. The portable power supply 101 can be any suitable form of a power storage device, such as one or more batteries. The battery can be any suitable battery type such as a lead acid battery or a lithium-ion battery or any anther suitable rechargeable and non-rechargeable batteries and combinations thereof.

In at least one implementation, the portable power supply 101 can power the pump 103 to provide continuous operation for up to about 8 hours or more. The portable power supply 101 and pump 103 can be combined in a single package or, as shown in FIG. 3, in separate packaging. In at least one implementation, the pressurized air source 105 can be held in a suitable packaging such as a fabric pack, not shown, capable of being secured to a belt or an arm or a leg or carried in a wearer's clothing pocket. The pressurized air source 105 can be portable and carried by a wearer. In one implementation, the pressurized air source 105 can include one or more mounting systems so that the pressurized air source can be backpack mounted, belt mounted or hand carried or any other suitable type of portability.

In at least one implementation, the filter 110 can be mounted directly to the pressurized air source 105. In another implementation, the filter 110 can be mounted directly to the mask 120. The filter media 111 can include a PTFE (polytetrafluoroethylene) filter media or other suitable filter media. In at least one implementation, the filter media 111 has a porosity of not more than about 1.2 micron, as 1.2 micron is recognized as sufficiently small enough to capture virus particles. The filter media can optionally have a porosity of less than about 1.2 micron (e.g., between about 0.5 and about 1.2 micron). In another implementation, the filter media can have a porosity of greater than 1.2 micron (e.g., between about 1.2 and about 3 micron) sufficient to capture particles larger than the respective selected porosity of the filter media.

The mask 120 can include a conformal shape that can conform to and substantially seal to a wearer's face sufficiently to ensure that substantially 100 percent of the air the wearer inhales is supplied from the filter 110. The mask 120 can optionally include one or more one-way outlet valves 123 capable of allowing the wearer's exhaled breath to escape from the mask 120 through the outlet valve(s). The mask 120 also include one or more mounting systems 121, such as straps and/or elastic materials, for securing the mask to a wearer's face and head.

The filtered air system 100 can also include a bag or case 130 for carrying the all of the system components. When in use, the case 130 can carry the pressurized air source 105. The case 130 can be secured to a wearer's waist, arm, or slung over the shoulder using the straps 132. The case 130 can include multiple pockets or compartments 134A, 134B.

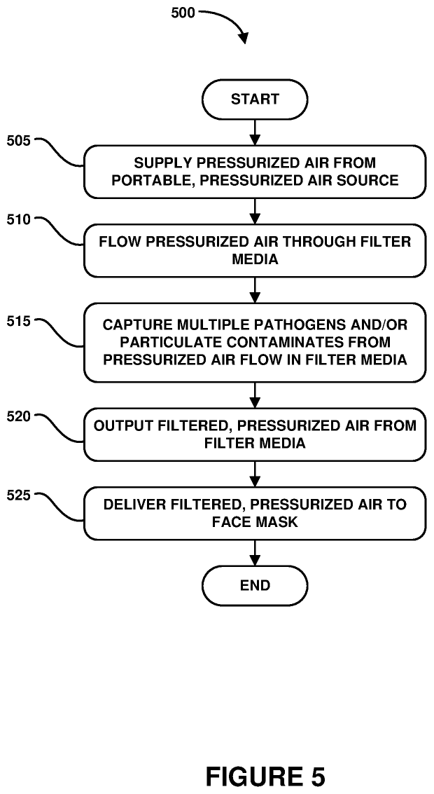

FIG. 5 is a flowchart diagram that illustrates the method operations 500 performed in removing pathogens and particulates in a portable air supply for implementing embodiments of the present disclosure. The operations illustrated herein are by way of example, as it should be understood that some operations may have sub-operations and in other instances, certain operations described herein may not be included in the illustrated operations. With this in mind, the method and operations 400 will now be described.

In an operation 505, pressurized air is supplied from the portable, pressurized air source 105.

In an operation 510, the pressurized air is directed to flow through the filter media 111 in the filter 110.

In an operation, 515, the filter media 111 captures one or more pathogens and particulate contaminates from the pressurized air.

In an operation 520, the filter 110 outputs filtered, pressurized air.

In an operation 525, the filtered, pressurized air is delivered to the face mask at an airflow rate and pressure that does not restrict the wearer's ability to inhale. In at least one implementation, the filtered, pressurized air has a pressure equal to or greater than atmospheric pressure and an airflow rate equal to or greater than the wearer's inhalation rate. Airflow in excess of the wearer's inhalation is vented out of the face mask through one or more one-way outlet valves 123 included in the face mask and/or escaping from a portion of the seal between the wearer's face and the face mask.

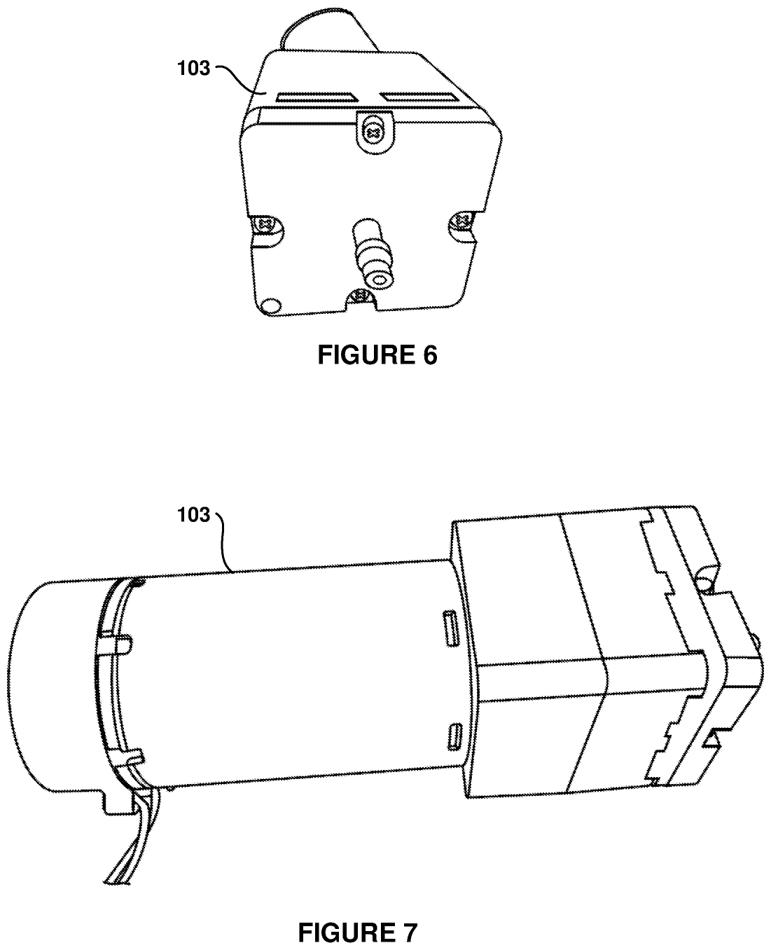

FIGS. 6 and 7 are more detailed pictorial views of an example pump 103, for implementing embodiments of the present disclosure. The pump 103 has an airflow rate between about 10 to about 15 liters per minute into the mask. In at least one implantation, the pump 103 is a diaphragm type pump, however, it should be understood that any suitable type of pump could be used. The pump 103 has a flow rate of between about 20 liters per minute as that is sufficient to most human inhalation rate, however, a higher or a lower flow rate pump can be utilized if desired so as to support the specific demands on the wearer. In at least one implantation, the pump 103 is powered by a direct current (DC) voltage provided by the power supply 101. The selected power supply can be any suitable voltage from about 3 volts DC to about 24 volts DC, with the pump 103 having a corresponding input voltage. By way of example, in one implementation, the power supply is a 5 volt DC power supply including one or more rechargeable batteries and a recharging circuit capable of being recharged by a universal serial bus (USB) port. Similar rechargeable battery packs including one or more rechargeable batteries can be utilized to form battery packs between about 3 and about 24 volts DC. In another implantation, the power supply can be about 12 volts DC to be compatible with numerous other 12 volt dc applications, e.g., automotive 12 volt DC systems such that the rechargeable batteries can be recharged from any suitable 12 volt DC recharging source (e.g., typically about 12.5-14.5 volts DC). The power supply 101 can also include one or more disposable batteries, such as typical lead acid and alkaline batteries, configured to form the desired voltage for the pump 103.

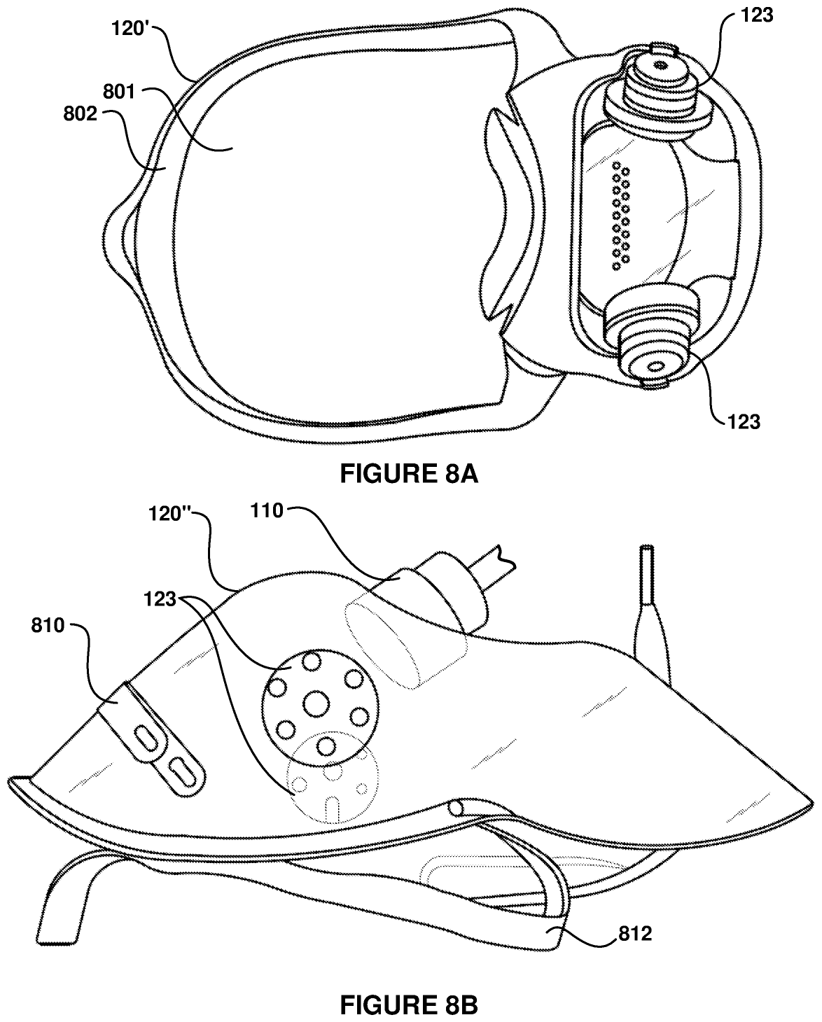

FIG. 8A is an example mask 120′, for implementing embodiments of the present disclosure. The mask 120′ includes a face shield 801 that covers the wearer's face having a seal 802 that forms a substantially air tight seal to at least a portion of a perimeter of the wearer's face. The face shield 801 provides protection for the wearer's eyes and face from exposure to particles and pathogens in the air.

FIG. 8B is a side view of another example mask 120″, for implementing embodiments of the present disclosure. The mask 120″ includes one or more one-way outlet valves 123. The mask 120″ includes the filter 110 mounted on the mask. The mask 120″ also includes straps 812 for securing the mask on the wearer's head and face. The mask 120″ or any other mask described herein, can optionally include one or more conformal strips 810 for forming the mask to the wearer's face. As shown, the conformal strip 810 can be a metallic strip that can be bent to match the contours of the wearer's nose. Other conformal strips, not shown, could be included to help conform the mask to the wearer's jawline, cheek and other portions of the wearer's face. When placed over the wearer's nose and mouth, the mask can form a substantially air tight seal between a perimeter of the mask and at least a portion of the wearer's face.

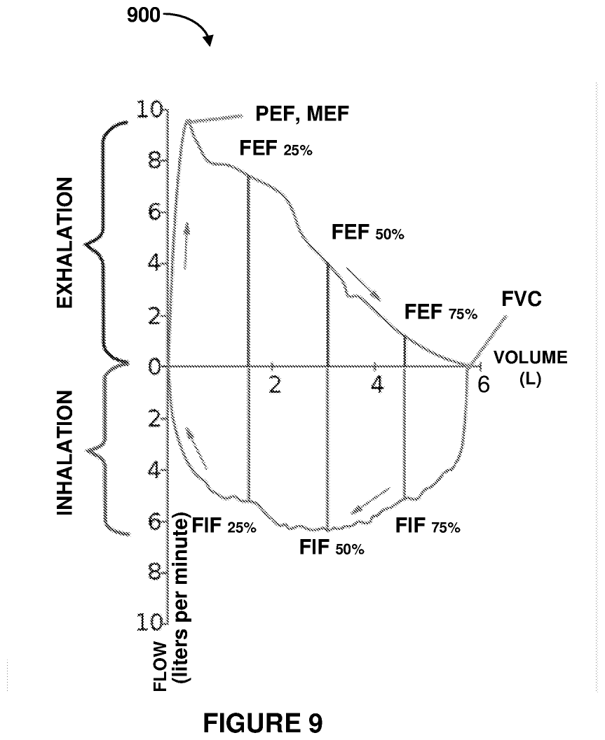

FIG. 9 is a graph 900 showing the airflow rate during a wearer's typical inhalation and exhalation, for implementing embodiments of the present disclosure. The flow-volume loop shows successful forced vital capacity (FVC) maneuver. Positive values represent exhalation by the wearer. Negative values represent inhalation by the wearer. At the start of the test, both flow and volume are equal to zero (representing the volume in the spirometer. As shown in the graph, typical wearer's inhalation and exhalation varies from 0 airflow rate to about 6 liters per minute.

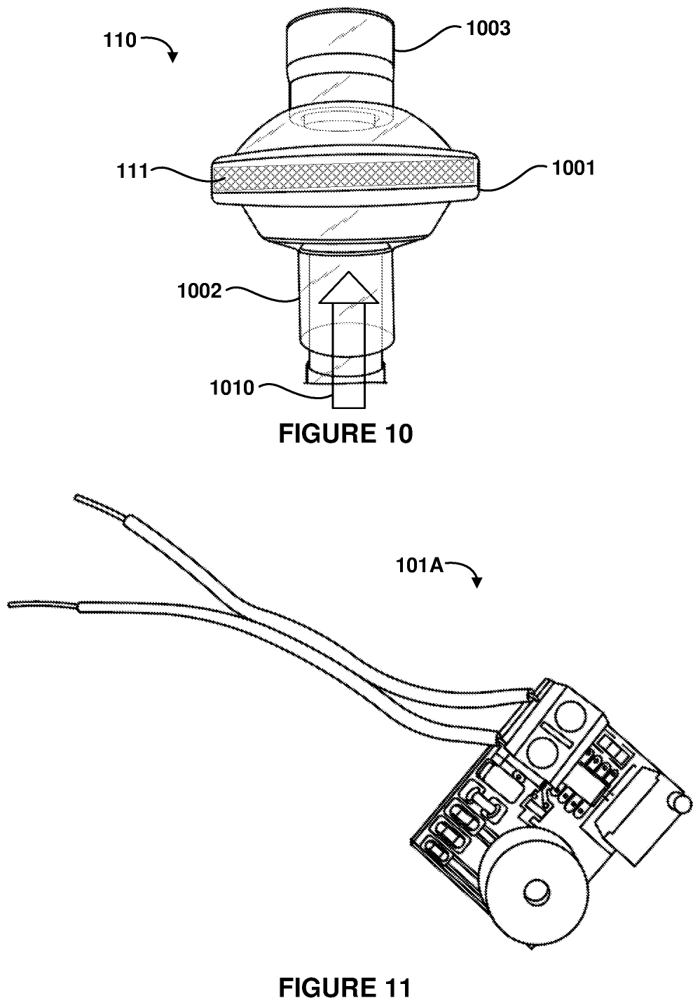

FIG. 10 is an example filter 110, for implementing embodiments of the present disclosure. The filter includes an outer shell 1001. The outer shell 1001 includes a quantity of filter media 111. The outer shell includes an inlet 1002 and an outlet 1003. Air flows through the filter 110 in direction 1010, into the inlet 1002, through the filter media 111 and out the outlet 1003. The portable pressurized air source 105 is fluidly coupled to the filter 110 by a first conduit 107 coupled to the inlet 1002. The filter 110 is fluidly coupled to the mask 120 by a second conduit 115 coupled to the outlet 1003. The outer shell 1001 can be any suitable shape.

FIG. 11 is an optional low battery alarm circuit 101A, for implementing embodiments of the present disclosure. The low battery alarm circuit 101A can optionally be coupled to power supply 101. The low battery alarm circuit 101A monitors a voltage available of a battery in the power supply 101. When the low battery alarm circuit 101A detects a voltage below a selected threshold voltage, an alarm indication is output from the low battery alarm circuit. The alarm indication can be one or more of an audible alarm or a visual indicator, such as a flashing indicator or a haptic indicator such as a vibrator. The alarm indication notifies the wearer the battery voltage has dropped below the selected threshold value and thus indicates the battery needs to be recharged or replaced.

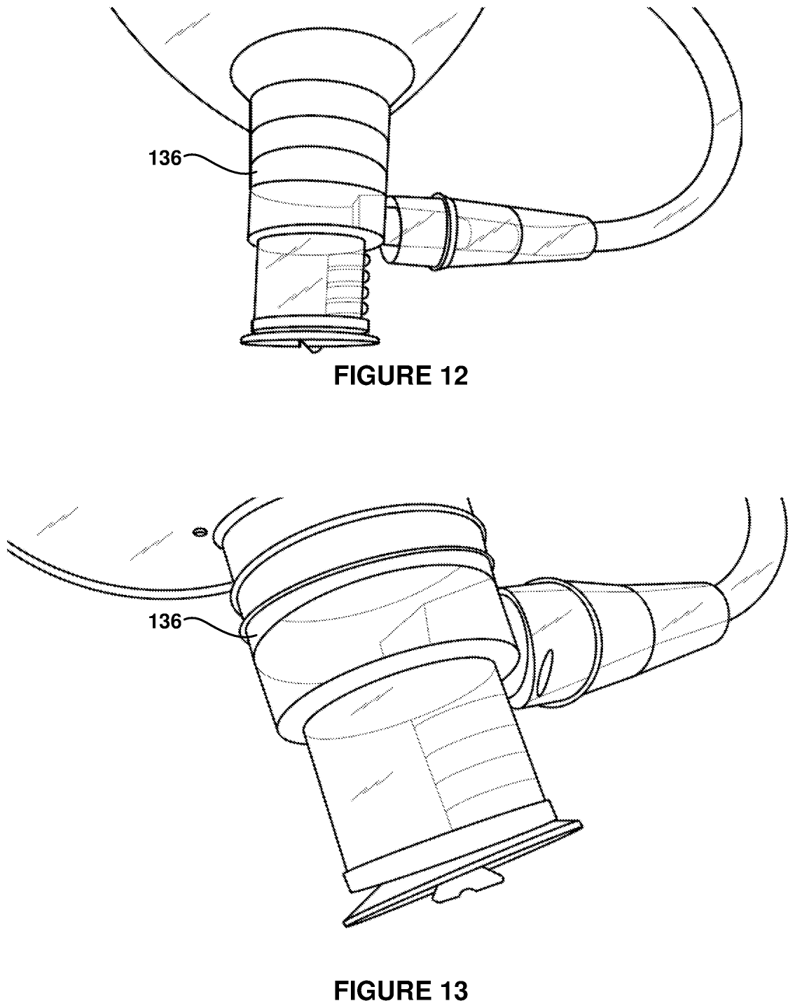

FIGS. 12 and 13 are an example check/pressure relief valve 136, for implementing embodiments of the present disclosure. The check valve 136 can be mounted on the mask or inline in the second conduit 115. The check valve 136 allows a wearer to exhale without fighting against the pressure of the portable pressurized air source 105. The check valve 136 can act as a one one-way valve capable of venting an excess pressure within the mask to the atmosphere out of the mask. The excess pressure could occur when the wearer exhales or if the pressurized, filtered airflow pressure exceeds a preselected pressure. The pressure to activate the one-way valve is typically slightly higher than atmospheric pressure (e.g., less than 2 psi greater than atmospheric pressure and in some implementations, less than 0.5 psi greater than atmospheric pressure). In at least one implementation, the check valve includes reed type valve element that opens toward the wearer side of the check valve as the wearer inhales and closes when the wearer exhales. The reed type element can be rubber, silicon, plastic or any other suitable flexible material.

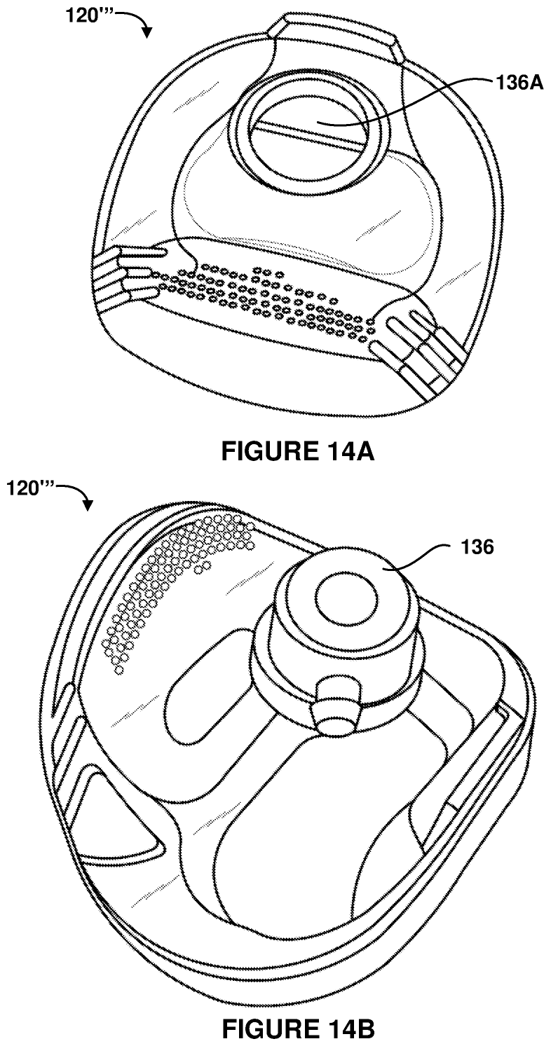

FIGS. 14A and 14B are an example mask 120′, for implementing embodiments of the present disclosure. FIG. 14A shows in inner view of the mask 120′ and FIG. 14B shows an outer view of the mask 120′″. The check/pressure relief valve 136 is mounted on the mask 120′ and includes a pressure relief element 136A. The pressure relief valve element 136A can be any suitable type of valve. In one implementation, pressure relief element 136A can be a reed type valve or a flexible disk type valve. The pressure relief valve element 136A can be removable for servicing, e.g., replacement and or cleaning.

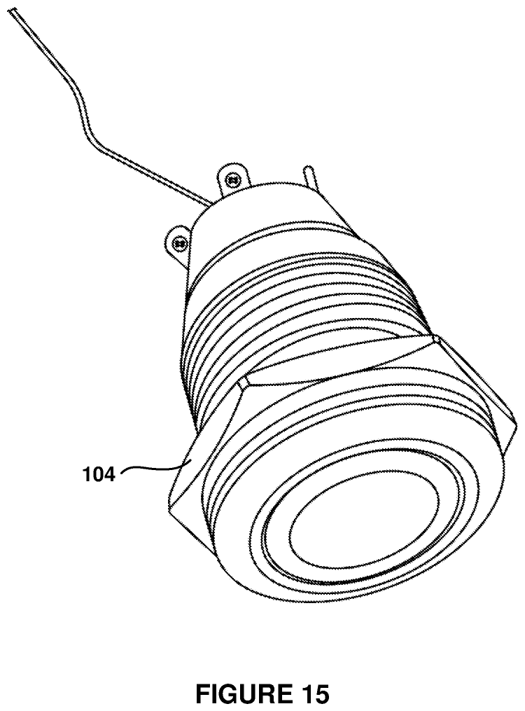

FIG. 15 is an optional, lighted, power switch 104, for implementing embodiments of the present disclosure. The power switch 104 can be coupled between the portable pressurized air source 105 and the power supply 101 for controlling power to the portable pressurized air source. The optional light included in the power switch 104 can provide an indication that the power is applied to the and indicating the portable pressurized air source 105.

Although the foregoing disclosure has been described in some detail for purposes of clarity of understanding, it will be apparent that certain changes and modifications may be practiced within the scope of the appended claims. Accordingly, the present embodiments are to be considered as illustrative and not restrictive, and the disclosure is not to be limited to the details given herein, but may be modified within the scope and equivalents of the appended claims.

What is claimed is:

1. A method of removing pathogens and particulates in a breathable air supply comprising:
supplying pressurized air from a portable pressurized air source;
flowing the pressurized air through a filter media including capturing a plurality of pathogens and particulate contaminates from the pressurized air to output filtered, pressurized air; and
delivering the filtered, pressurized air to a mask.

2. The method of claim 1, wherein delivering the filtered, pressurized air to the mask includes placing the mask over a wearer's mouth and nose.

3. The method of claim 2, wherein placing the mask over the wearer's mouth and nose includes forming a substantially air tight seal between a perimeter of the mask and at least a portion of the wearer's face.

4. The method of claim 1, wherein capturing the plurality of pathogens and particulate contaminates from the pressurized air includes capturing 99.9 percent of the pathogens in the pressurized air.

5. The method of claim 1, wherein the filter media has a porosity of less than about 1.2 micron.

6. The method of claim 1, wherein at least a first portion of the filter media includes polytetrafluoroethylene filter media.

7. A system for removing pathogens and particulates in an air supply comprising:
a portable pressurized air source;
a filter fluidly coupled to the portable pressurized air source, the filter capable of capturing a plurality of pathogens and particulate contaminates from a pressurized air flow from the portable pressurized air source and outputting a filtered, pressurized air flow; and
a mask fluidly coupled to the filter, the mask capable of forming a substantially air tight seal around a wearer's mouth and nose.

8. The system of claim 7, wherein the mask includes a face shield having capable of forming a substantially air tight seal between the mask and at least a portion of the wearer's face.

9. The system of claim 7, wherein the mask includes at least one one-way valve capable of venting an excess pressure within the mask.

10. The system of claim 7, wherein the filter media is capable of capturing 99.9 percent of the plurality of pathogens and particulate contaminants in the pressurized air.

11. The system of claim 7, wherein the filter media has a porosity of less than about 1.2 micron.

12. The system of claim 7, wherein at least a first portion of the filter media includes polytetrafluoroethylene filter media.
